(19) United States
(12) Patent Application Publication
(10) Pub. No.: US 2016/0331214 A1
(43) Pub. Date: Nov. 17, 2016

(54) ENDOSCOPE

(71) Applicant: OLYMPUS CORPORATION, Tokyo (JP)

(72) Inventors: Kiwamu FUJITANI, Tokyo (JP); Tatsuhiko SUZUKI, Tokyo (JP); Motohiro KURODA, Tokyo (JP); Nobuaki AKUI, Tokyo (JP); Reisuke OSADA, Tokyo (JP); Chikayoshi MEGURO, Tokyo (JP)

(73) Assignee: OLYMPUS CORPORATION, Tokyo (JP)

(21) Appl. No.: 15/221,992

(22) Filed: Jul. 28, 2016

Related U.S. Application Data
(63) Continuation of application No. PCT/JP2015/071320, filed on Jul. 28, 2015.

(30) Foreign Application Priority Data
Sep. 25, 2014 (JP) 2014-195590

(51) Int. Cl. A61B 1/00; A61B 1/015; A61B 1/06; A61B 1/005; A61B 1/05; A61B 1/018; A61B 1/267

(52) U.S. Cl. CPC A61B 1/00114; A61B 1/00066; A61B 1/018; A61B 1/015; A61B 1/2676; A61B 1/0052; A61B 1/05; A61B 1/06; A61B 1/00117

(57) ABSTRACT

An endoscope includes: an operation portion including a grasping portion configured to be grasped by a user and having a first longitudinal axis; an insertion portion configured to be inserted into a subject, the insertion portion having a second longitudinal axis that has a predetermined acute angle with respect to the first longitudinal axis and extending from the operation portion at a position on a more distal end side with respect to a treatment instrument insertion portion provided at the grasping portion; and a cable including, at an extension end thereof, a connector portion configured to be detachably connected with an external device, the cable having a third longitudinal axis on an extended line of the first longitudinal axis and extending from a distal end side of the operation portion.

### CROSS REFERENCE TO RELATED APPLICATION

This application is a continuation application of PCT/JP2015/071320 filed on Jul. 28, 2015 and claims benefit of Japanese Application No. 2014-195590 filed in Japan on Sep. 25, 2014, the entire contents of which are incorporated herein by this reference.

### BACKGROUND OF THE INVENTION

1. Field of the Invention

The present invention relates to an endoscope including a cable configured to be connected with an external device.

2. Description of the Related Art

Conventionally, endoscopes configured to be able to be introduced into a subject or an object have been widely used in medical fields or industrial fields, for example, in order to observe the subject or object such as inside of a living body or structure which is difficult to be observed. Such endoscopes are provided with image pickup means, illumination means, and the like.

An endoscope is provided with a universal cord which is a cable to be connected with an external device for transmission or reception of signals to and from image pickup means, or for transmission of illumination light to illumination means. Note that, in a common endoscope, a universal cord is extended from the side portion of the upper portion which is the proximal end side so as to be perpendicular to the longitudinal direction of the operation portion.

In contrast, Japanese Patent Application Laid-Open Publication No. 2002-58629 discloses the electronic endoscope configured such that the insertion portion is provided so as to extend in the direction which is substantially coaxial with the longitudinal direction of the operation portion, and the universal cord is extended so as to intersect with the insertion portion at an acute angle, for example.

### SUMMARY OF THE INVENTION

An endoscope according to one aspect the present invention includes: an operation portion including a grasping portion configured to be grasped by a user and having a first longitudinal axis; an insertion portion configured to be inserted into a subject, the insertion portion having a second longitudinal axis that has a predetermined acute angle with respect to the first longitudinal axis and extending from the operation portion at a position on a more distal end side with respect to a treatment instrument insertion portion provided at the grasping portion; and a cable including, at an extension end thereof, a connector portion configured to be detachably connected with an external device, the cable having a third longitudinal axis on an extended line of the first longitudinal axis and extending from a distal end side of the operation portion.

### BRIEF DESCRIPTION OF THE DRAWINGS

FIG. 1 is a perspective view showing a configuration of an electronic endoscope according to one aspect of the present invention.

FIG. 2 is a side view showing the configuration of the electronic endoscope according to the one aspect of the present invention.

### DETAILED DESCRIPTION OF THE PREFERRED EMBODIMENT

Hereinafter, description will be made on the present invention with reference to drawings.

Note that each of the drawings based on the embodiment below is a pattern diagram, and care should be taken to the fact that the relationship between the thicknesses and widths of the respective members, a ratio of the thickness of a certain member to that of another member, and the like are different from the actual ones, and there is a case where the respective drawings include parts in which the relationships and ratios among the dimensions are different.

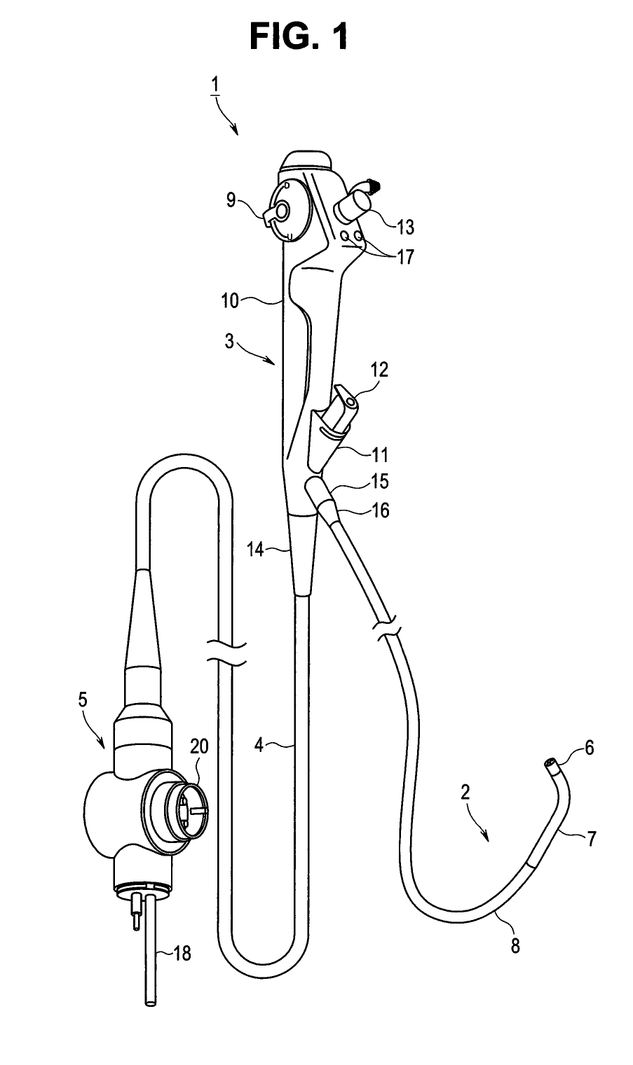

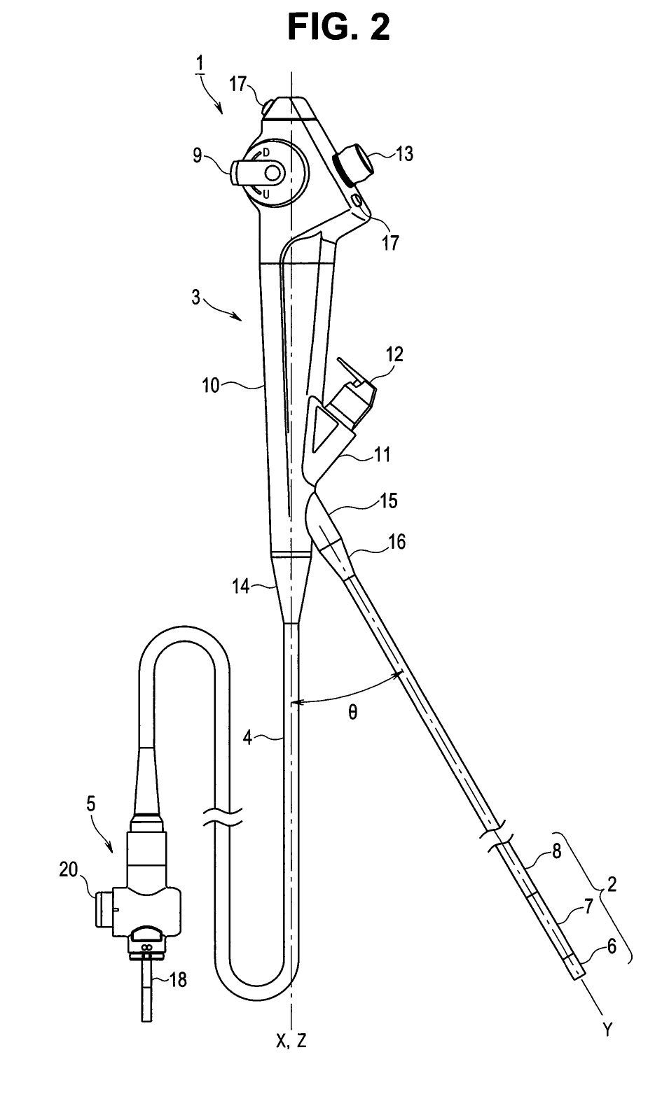

First, an embodiment of an endoscope according to one aspect of the present invention will be described below with reference to the drawings. The drawings relate to the present invention. FIG. 1 is a perspective view illustrating a configuration of an electronic endoscope, and FIG. 2 is a side view illustrating the configuration of the electronic endoscope.

In addition, description will be made below by taking a bronchoscope as an example, but the present invention is not limited to the bronchoscope, and the configuration of the present invention is applicable to various kinds of endoscopes.

As shown in FIGS. 1 and 2, an electronic endoscope (hereinafter, just referred to as endoscope) 1 according to the present embodiment is configured mainly by an insertion portion 2 formed in an elongated tube shape configured to be inserted into a subject, an operation portion 3 to which the proximal end of the insertion portion 2 is connected, a universal cord 4 which is an endoscope cable extended from the distal end side of the operation portion 3, and an endoscope connector 5 disposed at the distal end of the universal cord 4.

The insertion portion 2 is a flexible tubular member including a distal end portion 6, a bending portion 7, and a flexible tube portion 8 which are formed continuously in this order from the distal end side. Among these portions, in the distal end portion 6, an image pickup unit, which is an image pickup apparatus including inside thereof image pickup means, illumination means, and the like are housed.

The bending portion 7 is a mechanism part configured to be able to actively bend in two directions, i.e., up and down directions (UP-DOWN) by rotating a bending lever 9, to be described later, which is one of operation means of the operation portion 3.

Note that the bending portion 7 is not limited to such a type, and may be configured to able to bend in four directions including also right and left directions in addition to the up and down directions (the whole circumferential direction around the axis, i.e., UP-DOWN/RIGHT-LEFT, by up, down, right, and left operations of the operation lever).

The flexible tube portion 8 is a tubular member having flexibility so as to be passively flexible. Inside the flexible tube portion 8, not only a treatment instrument insertion channel but also various kinds of signal lines extended from the image pickup unit incorporated in the distal end portion 6 to be further extended to the inside of the universal cord 4 via the operation portion 3, a light guide that guides illumination light from a light source apparatus to allow the illumination light to emit from an illumination window disposed in the distal end portion 6, and the like are inserted (none of these components in the flexible tube portion are shown in the drawings).

The operation portion 3 is configured mainly by a grasping portion 10 configured to be grasped by a user with the hand when using the endoscope 1, a treatment instrument insertion portion 11 disposed at the halfway portion on the anterior face side of the grasping portion 10, operation means for operating various kinds of endoscope functions, and a suction valve 13 disposed at the upper portion on the anterior face side of the grasping portion 10.

In addition, the operation portion 3 includes a bend preventing portion 14 which is provided on the distal end side of the operation portion 3 and connected to the universal cord 4 so as to cover the proximal end of the universal cord 4, and a bend preventing portion 16 which is connected to a cylindrical protrusion portion 15 provided below the treatment instrument insertion portion 11 on the anterior face side of the grasping portion 10, and which is connected to the flexible tube portion 8 of the insertion portion 2 so as to cover the proximal end of the flexible tube portion 8.

Note that the operation means provided at the operation portion 3 include, for example, the bending lever 9 that performs bending operation of the bending portion 7, the suction valve 13 for suction operation, and a plurality of operation members 17 for performing corresponding operations for the image pickup means, the illumination means, and the like.

The treatment instrument insertion portion 11 is a configuration part which includes a treatment instrument insertion port through which various kinds of treatment instruments (not shown) are inserted, and which communicates with the treatment instrument insertion channel through a branch member inside the operation portion 3. The treatment instrument insertion portion 11 is provided with a forceps plug 12 which is a lid member for opening and closing the treatment instrument insertion port and which is configured to be attachable and detachable to and from (changeable with respect to) the treatment instrument insertion portion 11.

The universal cord 4 is a composite cable through which the various signal lines that extend from the distal end portion 6 of the insertion portion 2, pass through the inside of the insertion portion 2, to reach the operation portion 3, and further extend from the operation portion 3, and the light guide that transmits light from the light source apparatus (not shown) are inserted.

The endoscope connector 5 includes, on the side face portion thereof, an electric connector portion 20 to which a signal cable that detachably connects a video processor (not shown) which is an external device to the endoscope, and a light source connector portion 18 that detachably connects the light source apparatus which is an external device to the endoscope.

As shown in FIG. 2, in the endoscope 1 according to the present embodiment, the universal cord 4 has a central axis (longitudinal axis) Z along the longitudinal axis X of the operation portion 3 (central axis X of the operation portion 3 in some cases) and extends downward from the distal end of the grasping portion 10 (vertically downward which is right under the grasping portion 10 in the state where the user grasps the grasping portion 10 of the operation portion 3, for example). That is, the universal cord 4 has the central axis (longitudinal axis) Z on the extended line of the longitudinal axis X of the operation portion 3.

Further, in the endoscope 1, the insertion portion 2 in the linear state extends such that the central axis (longitudinal axis) Y thereof is set in the direction in which the central axis Y intersects with the longitudinal axis X of the operation portion 3 which is the extending direction of the universal cord 4 at a predetermined acute angle θ smaller than 90 degrees.

Furthermore, the insertion portion 2 in the linear state has the central axis Y that extends from the grasping portion 10, and the central axis Y is set so as to intersect with the longitudinal axis X of the operation portion 3 at a predetermined acute angle θ in the direction of the front face of the grasping portion 10, the front face being separated from the rear face of the grasping portion 10 of the operation portion 3 to which the user is opposed when the user grasps the grasping portion 10 of the operation portion 3.

Note that the insertion portion 2 extends from a position on the lower side of the grasping portion 10 which is more distal end side than the treatment instrument insertion portion 11 on the front face of the grasping portion 10 of the operation portion 3, the position being juxtaposed with the treatment instrument insertion portion 11 along the longitudinal axis X of the operation portion 3.

The operation portion 3 includes, on the anterior face side thereof, the suction valve 13, the plurality of operation members 17 and the treatment instrument insertion portion 11, and the insertion portion 2 is extended forward from the position on the linear line along the longitudinal axis X of the operation portion 3.

In the endoscope 1 according to the present embodiment which is configured as described above, the universal cord 4 is disposed so as to extend from the lower side of the operation portion 3, and the insertion portion 2 extends in the direction in which the insertion portion 2 intersects with the extending direction of the universal cord 4 at the predetermined acute angle θ, to thereby prevent the universal cord 4 including at the extension end thereof the endoscope connector 5 connected with the external device from interfering with the user by the universal cord 4 being got entangled around the user during the endoscopic examination. As a result, routing of the universal cord 4 is facilitated.

As described above, the endoscope 1 is configured such that the routing of the universal cord 4 which is the cable to be connected with the external device is facilitated so as to prevent the universal cord 4 from interfering with the user.

Furthermore, the endoscope 1 is configured such that the insertion portion 2 extends forward when the user grasps the operation portion 3. That is, the endoscope 1 has an advantage in that it is easy for the user to bring the insertion portion 2 close to the lying subject and easy to perform endoscopic examination.

Note that the technique according to the present invention is not limited to the electronic endoscope 1, but is applicable to an endoscope using a relay lens, an image fiber, and the like.

The invention recited in the above-described embodiment is not limited to the embodiment, and various modifications are possible in the practical stage without departing from the gist of the invention.

What is claimed is:

1. An endoscope comprising:
an operation portion including a grasping portion configured to be grasped by a user and having a first longitudinal axis;
an insertion portion configured to be inserted into a subject, the insertion portion having a second longitudinal axis that has a predetermined acute angle with respect to the first longitudinal axis and extending from the operation portion at a position on a more distal end side with respect to a treatment instrument insertion portion provided at the grasping portion; and
a cable including, at an extension end thereof, a connector portion configured to be detachably connected with an external device, the cable having a third longitudinal axis on an extended line of the first longitudinal axis and extending from a distal end side of the operation portion.

2. The endoscope according to claim 1, wherein the insertion portion extends forward from a front face of the operation portion, the front face being separated from a rear face of the operation portion to which a user who grasps the grasping portion is opposed.

3. The endoscope according to claim 1, wherein
the grasping portion is provided with the treatment instrument insertion portion through which a treatment instrument is inserted, and
the insertion portion extends from the position on the more distal end side with respect to the treatment instrument insertion portion of the grasping portion.

4. The endoscope according to claim 3, wherein the insertion portion extends from a position juxtaposed with the treatment instrument insertion portion of the grasping portion in a direction along the first longitudinal axis of the operation portion.

5. The endoscope according to claim 4, wherein
the operation portion includes an operation member for operating a suction valve and endoscope functions, and
the insertion portion extends from a position juxtaposed with the treatment instrument insertion portion, the suction valve, and the operation member in the direction along the first longitudinal axis of the operation portion.
